(19) United States
(12) Patent Application Publication
(10) Pub. No.: US 2015/0182143 A1
(43) Pub. Date: Jul. 2, 2015

(54) MAGNETIC RESONANCE IMAGING DEVICE, DIAGNOSTIC ASSISTANCE SYSTEM, AND PROGRAM

(71) Applicant: Hitachi Medical Corporation, Tokyo (JP)

(72) Inventors: Satoshi Hirata, Tokyo (JP); Yoshihisa Soutome, Tokyo (JP)

(21) Appl. No.: 14/418,234

(22) PCT Filed: Jul. 23, 2013
(86) PCT No.: PCT/JP2013/069932

§ 371 Date: Jan. 29, 2015

(30) Foreign Application Priority Data
Jul. 31, 2012 (JP) 2012-170017

(51) Int. Cl. A61B 5/055; A61B 5/00; G01R 33/385; G01R 33/34; G01R 33/24; G01R 33/30

(52) U.S. Cl. CPC A61B 5/055; G01R 33/243; G01R 33/307; G01R 33/385; G01R 33/34; A61B 5/7267; A61B 5/7282; A61B 5/742; A61B 5/7246; A61B 5/7221; A61B 2576/00; A61B 2560/0475

(57) ABSTRACT

Provided is a data analysis technique that uses an MRS spectrum obtained by MRS measurement, enabling a simple and a highly precise diagnostic support. This technique determines similarity between a record of the spectrum database on a disease basis being created in advance, and analysis data of an unknown MRS spectrum being newly acquired, thereby presenting a candidate disease. Determination of the similarity employs, out of the analysis data, only the data including a reliability index that satisfies a predetermined condition, as to each predetermined feature item. Similarly, creation of the spectral database on a disease basis employs, out of the analysis data containing at least one MRS spectrum with a definitive diagnosis, only the data including the reliability index that satisfies a predetermined condition, as to each predetermined feature item.

### TECHNICAL FIELD

The present invention relates to a data analysis technique for supporting clinical diagnosis using a magnetic resonance spectrum that is measured through the use of a magnetic resonance imaging apparatus.

### BACKGROUND ART

A magnetic resonance imaging (hereinafter, abbreviated as “MRI”) apparatus configured to perform MRI imaging, irradiates a subject placed in a static magnetic field with a radio frequency magnetic field having a specific frequency, thereby exciting nuclear magnetization of an atomic nucleus in hydrogen or the like, contained in the subject, detects nuclear magnetic resonance signals generated from the subject after the excitation, and acquires physical and chemical information. In addition to the magnetic resonance imaging for creating an image from the nuclear magnetic resonance signals, measuring techniques using the MRI apparatus include a magnetic resonance spectroscopy (hereinafter, abbreviated as “MRS”) measurement, which makes use of a difference in resonance frequencies caused by a chemical coupling between various molecules containing hydrogen nucleus (hereinafter, referred to as “chemical shift”), so as to separate nuclear magnetic resonance signals obtained from one to several regions, into signals on a molecular basis, thereby acquiring information of metabolites (see patent document 1, for instance).

The measuring method described in the patent document 1 is referred to as the “PRESS method”, which localizes a volume of interest, and it is the most frequently used method in the MRS measurement at present. In this PRESS method, after applying a gradient magnetic field (GC) pulse for selecting a predetermined slice, together with a radio frequency magnetic field (RF) pulse for exciting nuclear magnetization, gradient magnetic field pulses for selecting slices in two directions being orthogonal to the predetermined slice are applied respectively, together with an RF pulse for inverting the nuclear magnetization, and thereafter, measuring a nuclear magnetic resonance signal from a region where the three slices intersect with one another. Then, the nuclear magnetic resonance signals thus measured are subjected to the Fourier transform in the time axis direction, and magnetic resonance spectral signals are obtained.

The MRS measurement has a significant advantage that it is capable of measuring a metabolite inside a human body noninvasively, this advantage being incomparable with other measuring methods, and the MRS measurement is spreading gradually in the clinical field in recent years. However, since data obtained by the MRS is represented by a spectral graph, it is not easily interpreted, and experience is required, unlike a typical MRI image diagnosis. Therefore, the MRS measurement is recognized as a somewhat high-level diagnostic method for a doctor whose field of expertise is different. As an example of the clinical diagnosis to which the MRS is actually applied, there is suggested a threshold for determining (categorizing) an intensity value ratio of spectral signals (a concentration value ratio of major metabolites) on every disease, but this is not sufficient.

In view of this situation, in the project for studying clinical utility of the MRS in the Japanese Society for Magnetic Resonance in Medicine, set up by specialists of the present field, there is a planning to develop a diagnostic guideline and construct a case database (hereinafter, abbreviated as “DB”), aiming at further dissemination/education concerning the MRS. The case DB is a collection of cases containing as accumulation data, texts describing diagnostic results written in the medical records, and a spectral graph images. In disseminating the MRS in clinical sites, this kind of diagnostic support utilizing the case DB has a significant role for the future.

As one example of the case DB, attempts are being made to statistically construct a DB of spectral graphs on a disease basis of a specific organ (disease spectrum DB) (e.g., see the Non Patent Document 1). By way of example, according to the report of the multicenter clinical research conducted in Europe from the year of 2000 to 2002, there are created average spectral graphs of two patterns; short TE and long TE, as to each disease, in association with thirteen types of degenerative diseases in a head portion (3 to 86 examples per disease), and those spectral graphs are categorized visually according to a predetermined reference value, so as to be registered into a disease spectrum DB.

In the report of the multicenter clinical research, there is suggested a method to support a user to extract from the disease spectrum DB, an average spectrum that is highly approximate and similar to a newly acquired spectrum indicating a disease being unknown, and display thus extracted average spectrum on the unknown spectrum in a superimposing manner, thereby supporting diagnosis. There is further suggested a method to display the average spectrum extracted by the user and the standard deviation between the average spectrum and a group of average spectra registered in association with the disease to which the average spectrum belongs, on the unknown spectrum in a superimposing manner, in the form of a band spectrum (in which the line width corresponds to the standard deviation).

As a method for analyzing the concentration value (signal intensity value) of each metabolite contained in the measured magnetic resonance spectral signals (measured spectrum), there is Linear Combination Model (LCM) method (e.g., see the Non Patent Document 2). Firstly, a group of phantoms containing a simple substance of each metabolite at a predetermined concentration are used to obtain magnetic resonance spectral signals with respect to each metabolite. Those magnetic resonance spectral signals are assumed as the standard spectrum for each metabolite. The standard spectra of the respective metabolites are each magnified by a factor and summed, thereby making a candidate identification spectrum. A factor is determined so that a difference between this candidate identification spectrum and the measured spectrum is minimized. According to the factor being determined, the concentration value (or signal intensity value of each signal peak) of each metabolite contained in the measured spectrum is obtained as a probability density function. At this time, a standard deviation rate (hereinafter, referred to as “% SD”) may also be obtained, being a percentage display value of the standard deviation of each sample value in thus obtained probability density function.

### PRIOR ART DOCUMENT

### Patent Document

### Patent Document 1

Japanese Unexamined Patent Application Publication No. 59-107246

### Non Patent Document

### Non Patent Document 1

A. R. Tate et al., NMR IN BIOMEDICINE 2006 19 p. 411-434

### Non Patent Document 2

S. W. Provencher, Magnetic Resonance in Medicine, 1993 30 p. 672-679

### DISCLOSURE OF THE INVENTION

### Problem to be Solved by the Invention

The disease spectrum DB described in the Non Patent Document 1 is created according to the following procedure. Firstly, plural specialists verify the data quality, regarding at least one magnetic resonance spectral signal with respect to each disease definitively diagnosed (hereinafter, referred to as “definitive spectrum”). Then, peak alignment and normalization are applied to the definitive spectrum being accepted, an average value and a standard deviation are calculated, and thereafter, the definitive spectrum is registered as an average spectral graph in association with the disease.

Since plural persons concern the judgment in creating the disease spectrum DB, the criterion may become ambiguous, failing to stabilize the quality of the created DB. This may hinder highly precise analysis. In addition, since a step of quality verification by specialists is imperative, a user is not allowed to update or customize the DB, causing a low degree of flexibility. In addition, the time required for constructing the DB may be extended.

Following technique is used to extract an average spectrum that shows high similarity to the unknown spectrum. Simultaneously with creating the disease spectrum DB, a two-dimensional map is made by mapping on a plane, an attribute of each definitive spectrum that is used for creating the DB. As the attribute, a ratio between predetermined signal peaks is employed, for instance. A similar attribute is extracted from the newly acquired unknown spectrum, and the attribute is subject to the mapping process on the two-dimensional map. A user is allowed to recognize as having high similarity, the definitive spectrum that is mapped in proximity to the position on which the unknown spectrum is mapped, and thus extract the recognized definitive spectrum as the average spectrum to be displayed in a superimposing manner.

In order to extract the spectrum with high similarity, a ratio between signal peaks is employed, which is common to all the diseases. In nature, signals being generated vary disease by disease, and thus with regard to some diseases, the signal intensity of the signal peak used as the attribute may be weak, and this may reduce a reliability of the extraction.

The present invention has been made in view of the situation above, and an object of the present invention is to provide a data analysis technique that uses an MRS spectrum obtained by the MRS measurement, and enables a simple and highly precise diagnostic support.

### Means to Solve the Problem

The present invention is directed to determining similarity between a record of the spectrum database on a disease basis being created in advance, and analysis data of an unknown MRS spectrum being newly acquired, thereby presenting a candidate disease. Determination of the similarity employs, out of the analysis data, only the data including a reliability index that satisfies a predetermined condition, as to each predetermined feature item. Similarly, creation of the spectral database on a disease basis employs, out of the analysis data containing at least one MRS spectrum with a definitive diagnosis, only the data including the reliability index that satisfies a predetermined condition, as to each predetermined feature item.

### Effect of the Invention

An MRS spectrum obtained by the MRS measurement is used to perform simple and highly precise diagnostic support.

### BRIEF DESCRIPTION OF DRAWINGS

FIG. 1A illustrates an MRI apparatus according to an embodiment of the present invention, being an external view of the MRI apparatus of the horizontal magnetic field type;

FIG. 1B is an external view of the MRI apparatus of the vertical magnetic field type;

FIG. 1 C is an external view of the MRI device being enhanced in a sense of openness;

FIG. 2 is a block diagram showing a functional configuration of the MRI apparatus according to an embodiment of the present invention;

FIG. 3 illustrates one example of the pulse sequence of the PRESS method;

FIG. 4A illustrates a region which is excited by the pulse sequence according to the PRESS method;

FIG. 4B illustrates a region which is excited by the pulse sequence according to the PRESS method;

FIG. 4C illustrates a region which is excited by the pulse sequence according to the PRESS method;

FIG. 5 is a functional block diagram of the computer according to an embodiment of the present invention;

FIG. 6 is a flowchart showing a process for creating the disease spectrum DB according to an embodiment of the present invention;

FIG. 7A illustrates the disease spectrum DB according to an embodiment of the present invention;

FIG. 7B illustrates the measured analysis data according to an embodiment of the present invention;

FIG. 8 is a flowchart showing a candidate disease extracting process according to an embodiment of the present invention;

FIG. 9 is a flowchart showing a similarity determination process according to an embodiment of the present invention; and

FIG. 10 illustrates a system configuration example according to an embodiment of the present invention.

### BEST MODE FOR CARRYING OUT THE INVENTION

Hereinafter, there will be explained an embodiment to which the present invention is applied, with reference to the drawings. In the entire drawings for illustrating the present embodiment, the constituents having the same function will be labeled the same, and tedious explanation will not be made. This embodiment may not restrict the scope of the present invention.

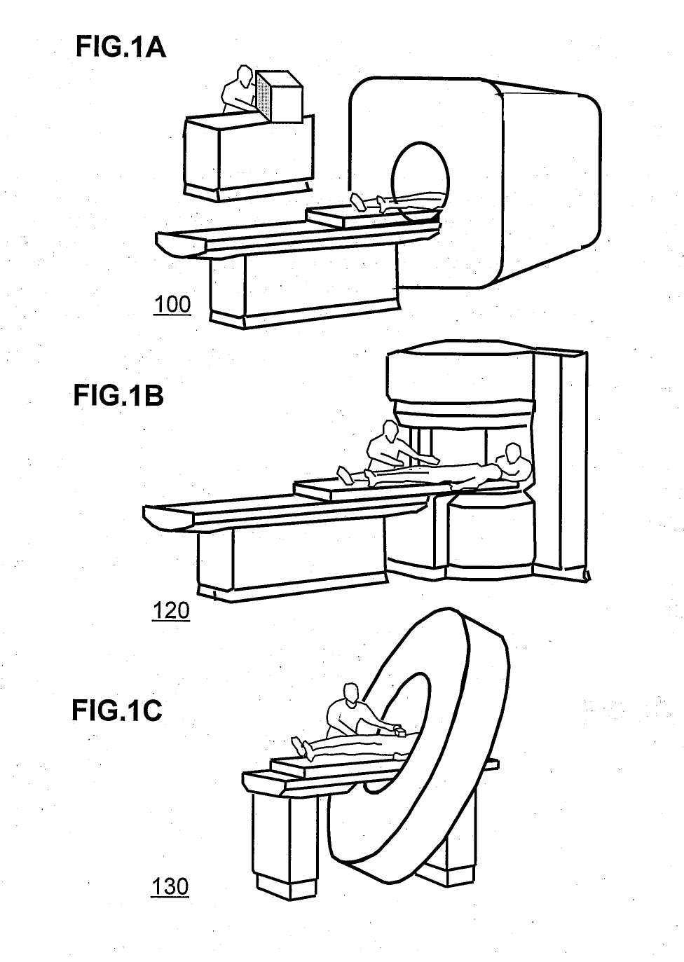

Firstly, the magnetic resonance imaging apparatus (MRI apparatus) of the present embodiment will be explained. FIG. 1 is an external view of an MRI apparatus according to the present embodiment. FIG. 1A illustrates the MRI apparatus of the horizontal magnetic field type 100 which uses a tunnel-type magnet for generating a static magnetic field by a solenoid coil. FIG. 1B illustrates the MRI apparatus 120 of a hamburger-type (open-type) vertical magnetic field system in which the magnets are separated vertically so as to enhance a sense of openness. FIG. 1C illustrates the MRI apparatus 130 of the same tunnel-type as that of FIG. 1A, using a magnet with reduced depth and put in a slanting position, thereby enhancing the sense of openness more. The present embodiment may employ any types of the MRI apparatus in those external views as shown above. It is to be noted that these are just a few examples, and the MRI apparatus of the present embodiment is not limited to those examples here. In the present embodiment, publicly known various MRI apparatuses may be employed, including any mode or any type thereof. Hereinafter, an explanation will be made, taking the MRI apparatus 100 as a representative example, unless otherwise distinguished.

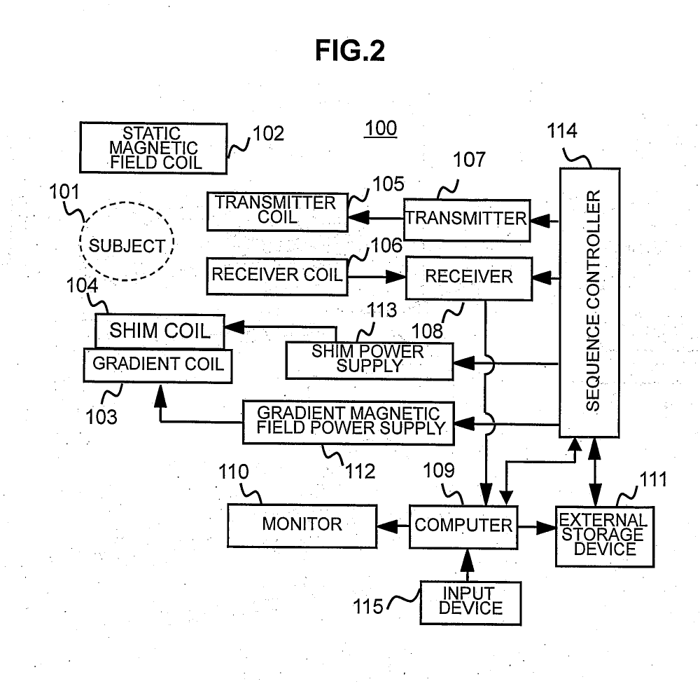

FIG. 2 is a functional block diagram of the MRI apparatus 100. As illustrated, the MRI apparatus 100 of the present embodiment is provided with a static magnetic field coil 102 configured to generate a static magnetic field in the space where the subject 101 is placed, a gradient coil 103 configured to generate a gradient magnetic field in each of the three directions (e.g., x-direction, y-direction, and z-direction) being orthogonal to one another, and apply the gradient magnetic fields to the subject 101, a shim coil 104 configured to adjust a static magnetic field distribution, a radio frequency magnetic field irradiation coil 105 (hereinafter, simply referred to as “transmitter coil”) configured to irradiate a measurement region of the subject 101 with the radio frequency magnetic field, a nuclear magnetic resonance signal receiver coil 106 (hereinafter, simply referred to as “receiver coil”) configured to receive a nuclear magnetic resonance signal generated from the subject 101, a transmitter 107, a receiver 108, a computer 109, a gradient magnetic field power supply 112, a shim power supply 113, and a sequence controller 114.

Various types of the static magnetic field coil 102 may be employed, depending on the structures of the MRI apparatuses 100, 120, and 130 as shown in FIG. 1A, FIG. 1B, and FIG. 1C, respectively. The gradient coil 103 and the shim coil 104 are driven by the gradient magnetic field power supply 112 and the shim power supply 113, respectively. The transmitter 107 generates the radio frequency magnetic field that is irradiated by the transmitter coil 105. The nuclear magnetic resonance signal detected by the receiver coil 106 is transferred to the computer 109 via the receiver 108. In the present embodiment, an explanation will be made taking as an example, the case where the transmitter coil 105 and the receiver coil 106 are separately provided. However, it is possible to configure such that one coil is employed which serves both functions, the transmitter coil 105 and the receiver coil 106.

The sequence controller 114 controls, according to instructions from the computer 109, the operations of the gradient magnetic field power supply 112 being a power source for driving the gradient coil 103, the shim power supply 113 being a power source for driving the shim coil 104, the transmitter 107, and the receiver 108, thereby controlling application of the gradient magnetic field and the radio frequency magnetic field, and receiving timing of the nuclear magnetic resonance signal. A time chart of the control is referred to as a pulse sequence, whose settings are previously configured depending on the measurement, and it is stored in a storage device, and the like, which is provided in the computer 109 as described below.

The computer 109 performs various computations on the nuclear magnetic resonance signals being received, so as to generate image information, spectrum information, temperature information, and temperature precision information, and provides an instruction to the sequence controller 114 to control the entire operations of the MRI apparatus 100. The computer 109 is an information processor provided with a CPU, a memory, a storage device, and the like, and the computer 109 is connected to the monitor 110 such as a display, an external storage device 111, and an input device 115.

The monitor 110 is an interface configured to display for an operator, a result and the like, being obtained by the computations. The input device 115 is an interface configured to allow the operator to input conditions, parameters, and the like, necessary for the computations performed in the present embodiment. The external storage device 111, along with the storage device, holds the data used in various computations executed by the computer 109, data obtained by the computations, conditions and parameters being inputted, and the like.

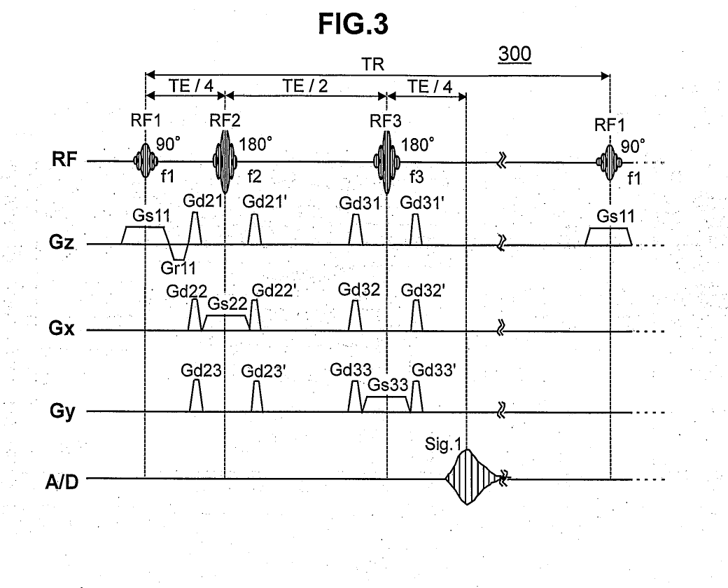

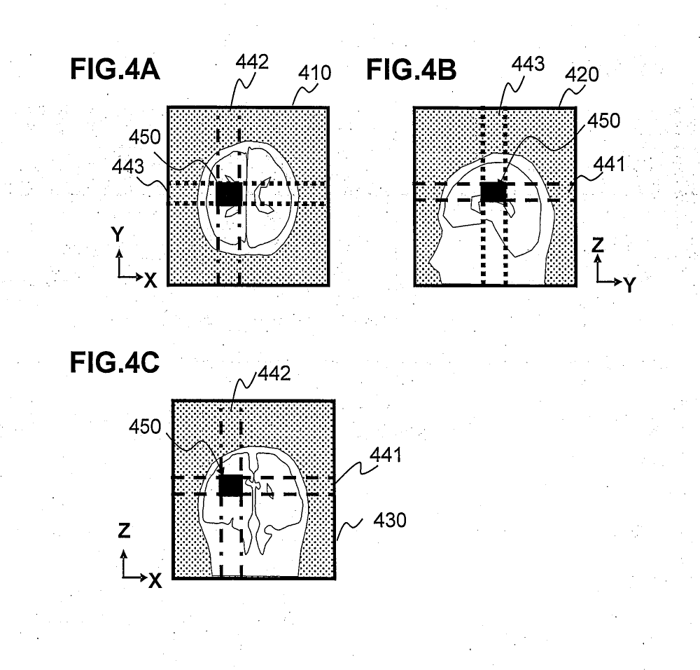

Next, an explanation will be provided as to the pulse sequence which is used in the MRS measurement according to the present embodiment. The present embodiment employs the symmetrical PRESS method being a basic measuring technique of the MRS measurement. With reference to FIG. 3 and FIG. 4, a relationship between the operations of each constituent element and a region to be excited according to the PRESS method will be explained.

FIG. 3 illustrates the pulse sequence 400 of the symmetrical PRESS method. Here, the MRI device 100 being the horizontal magnetic field system is employed, and the direction of the static magnetic field is assumed as the Z-axis direction. In the present pulse sequence 300, “RF” indicates a timing for applying a radio frequency magnetic field, “Gz” indicates a timing for applying a gradient magnetic field in the Z-axis direction, “Gx” indicates a timing for applying the gradient magnetic field in the X-axis direction, “Gy” indicates a timing for applying the gradient magnetic field in the Y-axis direction, and “A/D” indicates a timing for acquiring a nuclear magnetic resonance signal (echo signal). In addition, “TE” indicates an echo time.

FIG. 4 illustrates a region being excited and inverted by the pulse sequence 300 as shown in FIG. 3. It is to be noted that images as shown in FIG. 4 are scout images acquired for positioning and reference purposes prior to a main scan, and FIG. 4A shows a transverse image for positioning 410, FIG. 4B shows a sagittal image for position reference 420, and FIG. 4C shows a coronal image for position reference 430. In here, a region (voxel) 450 is assumed as a region targeted for the measurement, and a first slice 441 perpendicular to the Z axis, a second slice 442 perpendicular to the X axis, and a third slice 443 perpendicular to the Y axis intersect on the region (voxel) 450.

Firstly, a radio frequency magnetic field pulse RF1 at the flip angle 90° (90° pulse) is applied, together with applying a gradient pulse for selecting a slice (slice selective GC pulse) Gs11 in the Z axis direction, and only the nuclear magnetization within the first slice 441 is selectively rendered to be in the excitation state. In this situation, the transmit frequency f1 of the 90° pulse RF1 is determined in such a manner that the first slice 441 selected in combination with the slice selective GC pulse Gs11 contains the volume of interest 450. It is to be noted that as for all the radio frequency magnetic field pulses (RF pulses) in the following, it is possible to adjust each of the transmit frequency, excitation (inversion) frequency band, excitation (flip) angle, and transmission phase, allowing optional change of “the slice position and thickness” for performing the selective excitation/inversion, and “the angle and direction for tilting the nuclear magnetization” included in the selected slice.

Next, after a lapse of TE/4 from the application of the 90° pulse RF1, an RF pulse (180° pulse) at the flip angle 180° is applied together with applying the slice selective GC pulse Gs22 in the X axis direction, thereby rendering only the nuclear magnetization included in the second slice 442 to be inverted by 180°, out of the nuclear magnetization within the first slice 441 that is excited by the 90° pulse RF1. The transmit frequency f2 of the 1800 pulse RF2 is determined in such a manner that the second slice 442 selected in combination with the slice selective GC pulse Gs22 includes the volume of interest 450.

Furthermore, after a lapse of TE/2 from the application of 180° pulse RF2, an RF pulse (180° pulse) RF3 at the flip angle 180° is applied together with applying the slice selective GC pulse Gs33 in the Y axis direction, allowing 180° inversion again of only the nuclear magnetization within the volume of interest 450 included in the third slice 443, out of the nuclear magnetization in the region where the first slice 441 and the second slice 442 intersect, being inverted by the 180° pulse RF2. The transmit frequency f3 of the 180° pulse RF3 is determined in such a manner that the third slice 443 selected in combination with the slice selective GC pulse Gs33 includes the volume of interest 450.

According to the applications of those three pairs of the slice selective GC pulse and the region selective RF pulse, the inside of the volume of interest 450 is selectively excited, and a nuclear magnetic resonance signal Sig. 1 is generated from the volume of interest 450, assuming the point of time after the lapse of TE/4 from the application of the 180° pulse RF3 as the echo time. The nuclear magnetic resonance signal Sig. 1 being generated has signal variation in the time axis direction, and includes information of the aforementioned chemical shift. The receiver coil 106 detects this nuclear magnetic resonance signals Sig. 1 at predetermined sampling intervals, the computer 109 applies the Fourier transform to the signals in the time axis direction, and a magnetic resonance spectrum is obtained.

It is to be noted in the pulse sequence 300, the GC pulse Gr11 applied immediately after the application of the slice selective GC pulse Gs11 corresponds to a GC pulse (rephasing GC pulse) for rephasing the slice selective GC pulse Gs11. The GC pulse Gd21 and the GC pulse Gd21′, the GC pulse Gd22 and the GC pulse Gd22′, and the GC pulse Gd23 and the GC pulse Gd23′, being applied before and after applying the 180° pulse RF2, respectively, are GC pulses (dephasing GC pulses) which do not disturb the phase of the nuclear magnetization excited by applying the 90° pulse RF1, but perform dephasing (disturb the phase) only for the nuclear magnetization excited by applying the 180° pulse RF2, thereby reducing false signals. In addition, the GC pulse Gd31 and the GC pulse Gd31′, the GC pulse Gd32 and the GC pulse Gd32′, and the GC pulse Gd33 and the GC pulse Gd33′, being applied before and after applying the 180° pulse RF3, respectively, are GC pulses (dephasing GC pulses), which do not disturb the phase of the nuclear magnetization excited by applying the 90° pulse. RF1, but perform dephasing only for the nuclear magnetization excited by applying the 180° pulse RF3, thereby reducing false signals.

In the PRESS method, the pulse sequence 300 as described above is executed as the imaging sequence, thereby selectively exciting only the nuclear magnetization included in the volume of interest 450 where the three slices 441, 442, and 443 intersect as shown in FIG. 4, and it is possible to detect the nuclear magnetic resonance signal Sig. 1 generated from the volume of interest 450.

It is to be noted that if the integration of the signals is performed so as to obtain an SNR being required, the pulse sequence 300 is repeated at intervals of the repetition time TR, and the detection of the nuclear magnetic resonance signal Sig. 1 is repeated N times (typically, from a few dozen times to hundreds of times). In this situation, the entire measurement time becomes equal to “Repetition time×Integrating count=TR×N”. This repetition time TR is determined according to the time required for the excited magnetization to resume to thermally equilibrated state as before the excitation, and it varies depending on a type of the metabolite as a target for excitation, an irradiation RF strength (flip angle) for performing the excitation, and the like. In the case where the nuclear magnetization of a typical metabolite within a human body, being measurable by the MRS, is excited by the 90° pulse, the repetition time TR is set to be around from one to two seconds, in general.

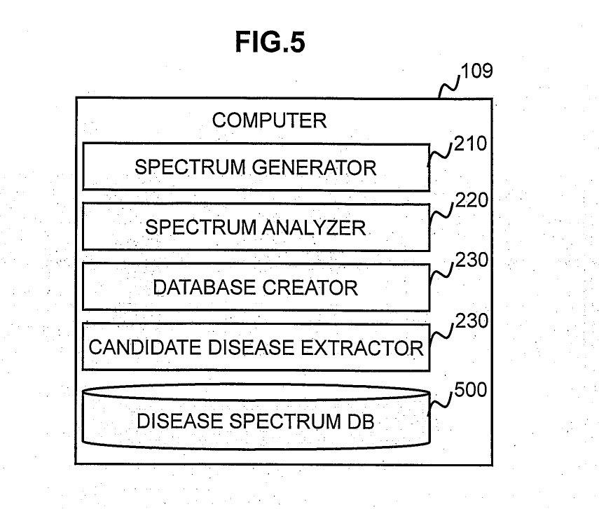

The MRI apparatus 100 of the present embodiment creates a database configured to store information indicating characteristics of the magnetic resonance spectrum with respect to each disease, without human intervention, and performs diagnostic support by using thus created database. An explanation will be provided as to the functions of the computer 109 of the present embodiment that implements this database creation and diagnostic support. FIG. 5 is a functional block diagram of the computer 109 according to the present embodiment. As illustrated, the computer 109 of the present embodiment is provided with a spectrum generator 210, a spectrum analyzer 220, a database (DB) creator 230, and a candidate disease extractor 240. The CPU in the computer 109 loads the programs stored in advance in the storage device into the memory, and executes the those programs, thereby implementing the functions above.

The spectrum generator 210 generated a magnetic resonance spectrum from the nuclear magnetic resonance signals received from the computer 109. In the present embodiment, for example, the nuclear magnetic resonance signal Sig. 1 obtained by executing the aforementioned pulse sequence 300 is subjected to Fourier transform in the time direction, thereby obtaining the magnetic resonance spectrum.

The spectrum analyzer 220 analyzes the magnetic resonance spectrum obtained in the spectrum generator 210, and calculates a predetermined characteristic value and a predetermined reliability index for each predetermined feature item, as analysis data. Calculation of the analysis data is performed by using the aforementioned LCM method, for instance.

The feature item may be a metabolite (a predetermined type of metabolite) included in the magnetic resonance spectrum, each signal peak of the magnetic resonance spectrum, or the like, for instance. The characteristic value may indicate at least one of a concentration value and a signal intensity value. Furthermore, in the case where the feature item is the metabolite, a standard deviation rate % SD is assumed as the reliability index. When the feature item is a signal peak, the signal to noise ratio SNR is assumed as the reliability index. The standard deviation rate % SD is calculated as a probability density function, being a percentage of the standard deviation of sample values of the characteristic value on a metabolite basis.

The standard deviation rate % SD represents that the smaller is the value calculated as the standard deviation rate % SD, the more probable is the characteristic value of the metabolite. The magnitude of the characteristic value is inversely proportional to the magnitude of the standard deviation rate % SD. The signal to noise ratio SNR is calculated by using the standard deviation of the peak area and the noise region.

The DB creator 230 creates the disease spectrum DB 500. The disease spectrum DB 500 is a database to store information (registered values) indicating a feature of the magnetic resonance spectrum on a disease basis, in the form of a record with respect to each disease. The disease spectrum DB 500 thus created is stored in the storage device that is provided in the computer 109.

The registered value of each record in the disease spectrum DB is calculated by using the analysis data (definitive analysis data) of one or more magnetic resonance spectra that are definitely diagnosed as showing a predetermined disease. In this case, the calculation is performed using only the characteristic value and the reliability index of the definitive analysis data in which the reliability index satisfies a predetermined condition. Timing for creating a record on a disease basis may be any time after at least a predetermined number of the definitive spectra are collected. Details of the creation will be described later.

The candidate disease extractor 240 uses the analysis data (measured analysis data) of the acquired magnetic resonance spectrum, determines a candidate disease that is estimated from the magnetic resonance spectrum, and presents the candidate disease to a user. The disease spectrum DB is used for this determination. A disease selected as the candidate disease is identified by the record that has a high degree of similarity to the measured analysis data. Similarity to the registered value of the feature item in each record of the disease spectrum DB may determine a degree of similarity. Details of the determination technique will be described later. It is to be noted that the name of each candidate disease is displayed on the monitor 110, for instance, thereby presented to the user.

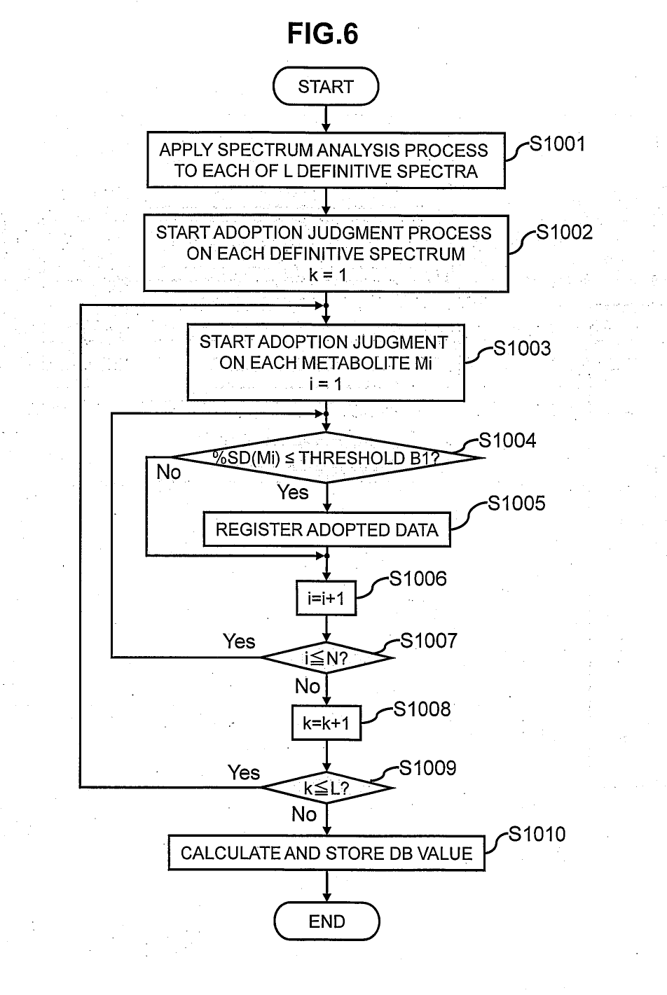

Next, details of the disease spectrum DB creation process according to the DB creator 230 will be explained, with reference to the flow thereof. FIG. 6 illustrates one example of the processing flow of the disease spectrum DB creation process according to the present embodiment. In here, a process for creating a record in association with one disease will be explained. The disease spectrum DB creation process of the present embodiment starts in response to an instruction from the user, after collecting a predetermined number of definitely diagnosed magnetic resonance spectra (definitive spectra) regarding the disease targeted for creation, as described above. In addition, prior to the disease spectrum DB creation process according to the DB creator 230, the spectrum analyzer 220 performs a process for analyzing the definitive spectrum. In the following processing flow, an explanation will be provided, including this analytical process according to the spectrum analyzer 220.

In this example here, the number of the definitive spectra being collected is assumed as L (L is an integer at least one). A metabolite is used as the feature item, a concentration value is used as the characteristic value, and a standard deviation rate % SD is used as the reliability index. The number of metabolites whose concentration value is calculated is assumed as N (N is an integer at least one).

Firstly, the spectrum analyzer 220 performs the spectral analysis process on each of the L definitive spectra (step S1001). Here, the spectrum analyzer calculates as the definitive analysis data, the concentration value and the standard deviation rate % SD of each metabolite, with respect to each of L definitive spectra. Consequently, L×N definitive analysis data items are calculated. Hereinafter, each metabolite is represented as Mi (i=1, 2 . . . N), and the standard deviation rate of each metabolite is represented as % SD (Mi).

Next, the DB creator 230 starts a process of adoption judgment on each definitive spectrum (step S1002). In the adoption judgment process, it is determined whether or not each of L×N definitive analysis data items is adopted for creating the record of the disease spectrum DB. Here, it is determined whether or not definitive analysis data of each metabolite Mi is adopted, with respect to each definitive spectrum being a source of analysis. Therefore, firstly, the counter k for counting the definitive spectrum is initialized (k=1).

Then, the DB creator 230 starts the adoption judgment on each metabolite Mi (step S1003). The adoption judgment process determines as to each metabolite Mi, reliability of a group of definitive analysis data items that are obtained from the k-th definitive spectrum, and it is determined whether or not the metabolite is adopted. In order to start the adoption judgment process, the DB creator 230 initializes the counter i (i=1) that counts the metabolite Mi.

In the adoption judgment process, the DB creator 230 compares the standard deviation rate % SD (Mi) within the definitive analysis data of each metabolite Mi, being calculated in the step S1001, with a predetermined threshold B1 (step S1004). As the threshold B1, the value of 20 is employed, for instance. Then, if the standard deviation rate % SD (Mi) is equal to or less than the threshold B1 (% SD≦B1), it is determined to adopt the definitive analysis data of the metabolite Mi, and it is registered in the storage device as adopted data (step S1005). The registration is performed establishing association with the definitive spectrum and the metabolite Mi. In other cases, the data is not registered.

The processes in the step S1004 and in the S1005 are repeatedly performed on all the metabolites Mi. In other words, the processes are repeated along with incrementing the counter i by one, until the counter i reaches N (steps S1006 and S1007).

Then, the DB creator 230 repeats the judgment process as to each metabolite Mi (the processes from step S1003 to step S1007), on all of the definitive spectra. In other words, the judgment process is repeated by incrementing the counter k by one, until the counter k reaches L (steps S1008 and S1009).

Thereafter, the DB creator 230 uses the definitive analysis data registered as adopted data to calculate a registered value of the record, and stores the registered value in the disease spectrum DB (step S1010) In the present embodiment, the registered value of the record is calculated on a metabolite Mi basis. Specifically, the data of the same metabolite Mi is extracted from the adopted data, statistical values of the concentration value and the standard deviation rate % SD are respectively calculated, as to each metabolite Mi, and the statistical values are stored as the registered values. The statistical values may be an average value and a variance value, for instance. The statistical value to be registered is not restricted to one type.

The spectrum analyzer 220 and the DB creator 230 of the present embodiment perform the aforementioned processes from the step S1001 to the step S1010, on the definitive spectrum indicating each disease, generate records on a disease basis, and construct the disease spectrum DB.

In the aforementioned step S1010, it is further possible to configure such that the DB creator 230 calculates, not only the registered value on a metabolite Mi basis, but also “averaged spectral waveform (average spectral waveform) and its standard deviation waveform”, by using all the adopted data, and registers the calculated results as the DB information.

When the average spectral waveform and the standard deviation waveform are calculated, the DB creator 230 firstly uses all the adopted spectral data (each signal intensity value) to calculate the “average value of the signal intensity” and “standard deviation of the signal intensity” at each point on the horizontal axis. A waveform obtained by connecting each point indicating the “average value” on all the horizontal axis points being calculated, corresponds to the average spectral waveform. A waveform obtained by connecting each point indicating a “value of sum of the average value and the standard value” on all the horizontal axis points, corresponds to the upper limit of the standard deviation waveform, and a waveform obtained by connecting each point indicating a “value of difference between the average value and the standard deviation” on all the horizontal axis points, corresponds to the lower limit of the standard deviation waveform.

In the aforementioned processing flow, all the definitive spectra being collected are analyzed in advance, then all the analysis data items are calculated, and thereafter, it is determined whether or not the analysis data is adopted. However, this procedure is not limited to this example. By way of example, if the analysis data of each feature item is able to be calculated independently from each definitive spectrum, it may be determined whether or not the analysis data is adopted, every time the analysis data is calculated on a feature item basis. It is further possible to configure such that the analysis data is calculated for each definitive spectrum, and determine whether or not the analysis data is adopted.

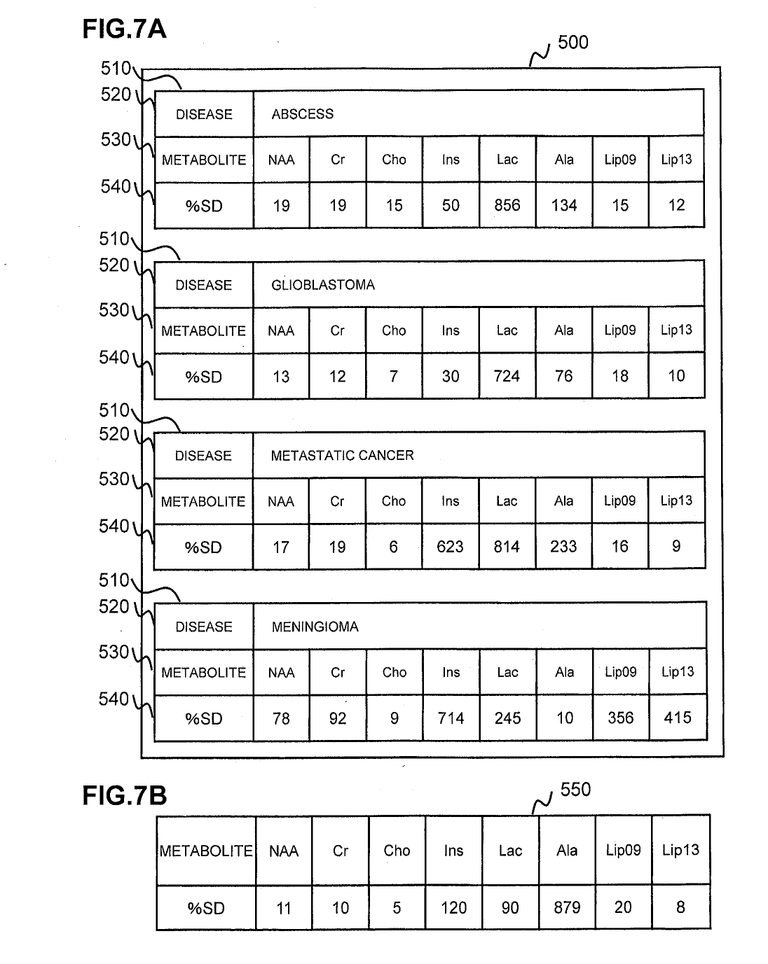

FIG. 7A illustrates an example of the disease spectrum DB 500 relating to a head portion, the DB being created by the DB creator 230 according to the procedure as described above. In this example here, there are shown the record 510 indicating that the disease is abscess, the record 510 indicating that the disease is glioblastoma, the record 510 indicating that disease is metastatic cancer, and the record 510 indicating that the disease is meningioma. As the registered value on a metabolite Mi basis, the standard deviation rate % SD is shown as a representative example. As illustrated, each record 510 in the disease spectrum DB 500 of the present embodiment is provided with the disease name 520 being information identifying the disease, the feature item (metabolite, in this example) 530, and the registered value 540 obtained from the reliability index (standard deviation rate % SD in this example) of each feature item 530. Though not illustrated, each characteristic value may also be stored as the registered value 540.

Here, in the disease spectrum DB creation process, if any of the standard deviation rate % SD (Mi) of one metabolite Mi obtained from L definitive spectra being prepared, does not reach the aforementioned reference (larger than B1), the adopted data of this metabolite Mi becomes zero. In this case, zero may be registered as the characteristic value of the metabolite Mi of this disease. If zero is registered, the user is allowed to know that there is no adopted data.

When there is no adopted data, it is possible to configure such that the registered value of the reliability index (standard deviation rate % SD in the example above) may be calculated by using a value of the definitive analysis data determined as not adopted, and then registered.

In addition, it is also possible to judge whether or not the definitive spectrum itself is accepted. By way of example, a metabolite that is to be subjected to the adoption judgment is designated disease by disease. Then, when it is determined that the metabolite to be subjected to the adoption judgment is determined to be all adopted, the definitive spectrum being a source of the definitive analysis data is accepted. It is further possible to calculate the registered value of the disease spectrum DB, using only the reliability index and the characteristic value obtained from the definitive spectrum determined as accepted.

The record 510 of the disease spectrum DB 500 already constructed may be updated by using a new definitive spectrum of the disease. In this case, firstly, the spectrum analyzer 220 calculates the characteristic value and the reliability index for each feature item of the definitive spectrum to be newly added. Then, on a feature item basis, the adoption judgment is performed by using the reliability index according to the technique of the step S1004. Subsequently, the registered values (characteristic value and the reliability index) 540 of the record 510 of the feature item that is determined as adopted are updated by its adopted data (the characteristic value and the reliability index).

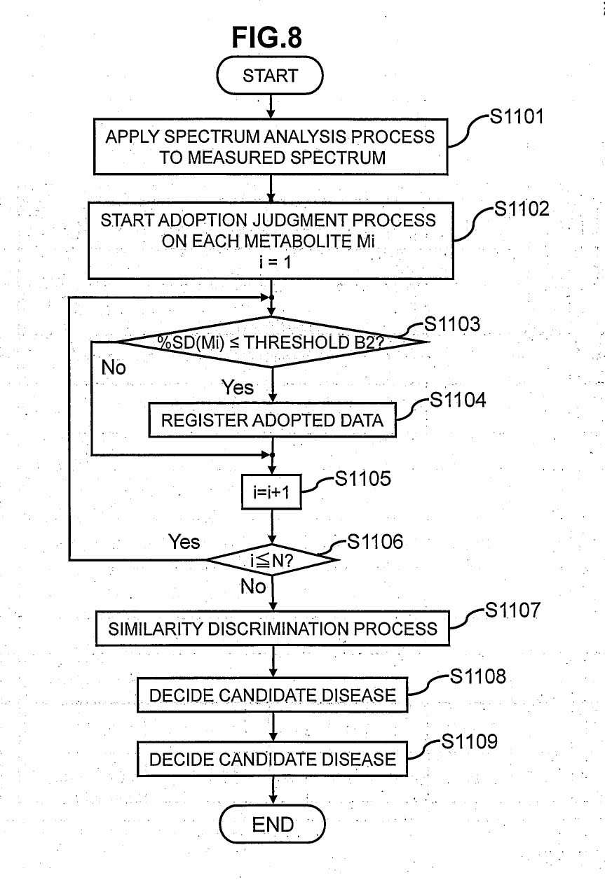

Next, the candidate disease extraction process according to the candidate disease extractor 240 will be explained. FIG. 8 is a flow of the candidate disease extraction process of the present embodiment. Starting of the candidate disease extraction process of the present embodiment is triggered, for example, by performing the imaging according to the aforementioned PRESS sequence and obtaining the magnetic resonance spectrum data of the patient. Hereinafter, the acquired magnetic resonance spectrum data being the analysis target is referred to as measured spectrum. Prior to the candidate disease extraction process according to the candidate disease extractor 240, the spectrum analyzer 220 performs the analytical process on the measured spectrum. In the following process flow, an explanation will be provided including this analytical process according to the spectrum analyzer 220.

Similar to the disease spectrum DB creation process, the concentration value is used as the characteristic value, the standard deviation rate % SD is used as the reliability index, and the number of metabolites being the analysis target is assumed as N. N metabolites are represented as Mi (i=1, 2, . . . N). Furthermore, the standard deviation rate % SD of the measured spectrum for each metabolite is represented as % SDs (Mi).

The spectrum analyzer 220 performs the spectral analysis process on the measured spectrum (step S1101). Here, as to the measured spectrum being obtained, the concentration value and the standard deviation rate % SDs (Mi) of each metabolite are calculated as the measured analysis data. Consequently, N measured analysis data items are obtained. FIG. 7B illustrates an example of the measured analysis data 550 that is obtained here.

The candidate disease extractor 240 starts the adoption judgment process on each metabolite Mi (step S1102). Here, the adoption judgment process is performed for determining with respect to each metabolite Mi, whether or not the measured analysis data of the metabolite Mi is to be adopted for similarity judgment. In order to start this process, firstly, the counter i for counting the metabolite Mi is initialized (i=1).

In the adoption judgment process, the candidate disease extractor 240 compares the standard deviation rate % SDs (Mi) of the metabolite Mi calculated in the step S1101 with the threshold B2 being predetermined (step S1103). The threshold B2 may be the same value as the threshold B1 used in the DB creation process, and it may be 20, for instance. If the standard deviation rate % SDs (Mi) is equal to or less than the threshold B2 (% SDs B2), it is determined the measured analysis data of the metabolite Mi is adopted in the similarity judgment, and it is registered in the storage device as the adopted data (step S1104). The measured analysis data is registered in association with the metabolite Mi. In other cases, this data is not registered.

The processes in the steps S1103 and S1104 are repeated for all the metabolites Mi. In other words, those processes are repeated by incrementing the counter i by one, until the counter i reaches N (steps S1105 and S1106).

Thereafter, the candidate disease extractor 240 uses each data registered as the adopted data to perform the similarity discrimination process for determining the similarity between the record 510 on a disease basis registered in the disease spectrum DB 500, and the registered data (step S1107). In the present embodiment, as described below, the similarity discrimination process calculates a similarity index as to each of the records 510 (disease) registered in the disease spectrum DB 500, in such a manner that the smaller value indicates the similarity index, the higher is the similarity, for instance.

Then, the candidate disease extractor 240 uses the similarity index being a result of the similarity discrimination process, so as to-determine the candidate disease (step S1108). In the present embodiment, a predetermined number of records 510 are extracted in the order from the record having the highest similarity, and the disease identified by the record 510 is assumed as the candidate disease.

Then, the candidate disease extractor 240 displays the information identifying the candidate disease on the monitor 110, and terminates the process (step S1109). In this situation, the disease name is displayed as the information identifying the candidate disease.

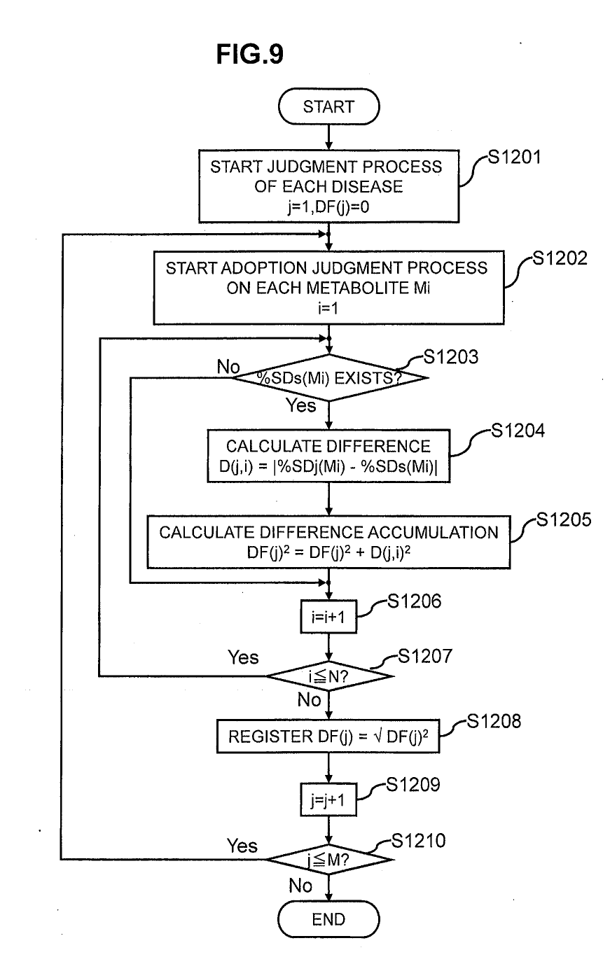

Next, details of the similarity discrimination process according to the candidate disease extractor 240 of the step S1107 will be explained. FIG. 9 is a flow of the similarity discrimination process according to the present embodiment. Hereinafter, it is assumed that in the disease spectrum DB 500, M types (M is an integer at least one) of diseases are registered as the records 510. It is further assumed that in each of the records 510, there is registered the reliability index for each of the N metabolites Mi (here, the reliability index is the standard deviation rate % SD (Mi)). In addition, the standard deviation rate of the metabolite Mi of the j-th record (disease j) (j is an integer between or equal to one and M) 510 is assumed as % SDj (Mi).

In the similarity discrimination process, the candidate disease extractor 240 calculates the index (similarity index) indicating the similarity to the measured analysis data, as to all the records 510 of the M types of diseases that are registered in the disease spectrum DB 500. In the present embodiment, a sum of difference DF(j) is used as the similarity index, the sum of difference being a positive value of the root-sum-square value of the difference between the registered value as to each metabolite Mi and the adopted data obtained in the step S1104. In the present embodiment, the standard deviation rate % SD is used as the registered value and the adopted data, for the calculation to obtain the difference therebetween.

The candidate disease extractor 240 starts the judgment process as to each disease (step S1201). Here, in order to start the judgment process with respect to each record 510, i.e., each disease in the disease spectrum DB 500, the counter j for counting the record 510 is initialized (j=1), and the sum of difference DF(j) relating to the registered value of the j-th record 510 is initialized (DF(j)=0).

Then, the candidate disease extractor 240 starts the adoption judgment as to each metabolite Mi (step S1201). Here, in order to start the difference calculation for each metabolite Mi, the counter i for counting the metabolite Mi is initialized (i=1).

Then, firstly, it is determined whether or not the standard deviation rate % SDs (Mi) of the metabolite Mi is registered in the adopted data (step S1203).

Then, if it is registered, the difference calculation is performed (step S1204). Specifically, as to the metabolite Mi of the j-th record 510 (disease j), a difference D(j, i) between the registered value (% SDj (Mi)) and a value registered as the adopted data (% SDs (Mi)) is calculated. In the present embodiment, the following formula (1) is used to calculate the reliability index:

D(j,i)=|% SDj(Mi)−% SDs(Mi)| (1)

Then, the square of the result is calculated and added to the square of the sum of difference DF(j), thereby updating the square of the sum of difference DF(j) (step S1205). Specifically, the calculation is performed according to the following formula (2):

DF(j)²=DF(j)²+D(j,i)² (2)

If it is determined that the standard deviation rate % SDs (Mi) of the metabolite Mi is not registered in the step S1203, the difference calculation in the step S1205 and the sum of difference updating in the step S1206 are not performed.

The processes from the step S1203 to the step S1205 are repeated for all the metabolites Mi. In other words, the processes are repeated by incrementing the counter i until the counter i reaches N (steps S1206 and S1207).

Then, the candidate disease extractor 240 calculates a positive value of the root of the square DF(j) of thus obtained sum of difference, and stores the result in the storage device, as the similarity index, in association with the disease j of the disease spectrum DB (step S1208).

Then, the candidate disease extractor 240 performs the aforementioned processes from the step S1202 to the step S1208, on all the records 510 of M types diseases registered in the disease spectrum DB 500, calculates the similarity index as to each record 510 (disease), registers those similarity indexes in the storage device (step S1209 and step S1210), and then terminates the process.

It is to be noted that in the candidate disease extraction process, upon displaying in the step S1109, the similarity index obtained in the similarity discrimination process in the step S1107 may be displayed together. Furthermore, in the case where the average spectral waveform and the standard deviation waveform are registered in the disease spectrum DB, those may be displayed together.

When displaying is performed in the step S1109 above, the candidate diseases are displayed in the order from the one with the highest similarity, but it is possible to configure such as listing the candidate diseases being extracted in no particular order.

It is to be noted that in this example, the similarity degree is determined using only the similarity index calculated from the reliability index, but this is not the only example. By way of example, the similarity index may be calculated from the characteristic value of each metabolite in the similar manner, and this similarity index may be used for determining the similarity. In addition, the similarity degree may be determined, considering a result of comparison of characteristic value ratios (concentration ratio, signal intensity value ratio, or the like) between the metabolites.

In the case where some diseases having a chance of similarity are extracted in advance, the similarity discrimination process may not be applied to all the diseases registered in the disease spectrum DB (all the records 510), but the possible diseases (records 510) only.

In the present embodiment, a metabolite is used as the feature item, and a standard deviation rate % SD is used as the reliability index, in the aforementioned disease spectrum DB creation, in the candidate disease extraction process, and in the similarity discrimination process, but this is not the only example. By way of example, a signal peak may be used as the feature item, and a signal-to-noise ratio SNR may be used as the reliability index.

In this case, instead of obtaining the standard deviation rate % SD as to each metabolite, the signal-to-ratio SNR may be calculated as to each signal peak. In addition, in the disease spectrum DB creation process, the processes from the step S1003 to the step S1007 are repeated, and in the candidate disease extraction process, the processes from the step S1102 to the step S1106 may be repeated, only by the number of the signal peaks.

At least one of the concentration value and the signal intensity value may be sufficient as the characteristic value.

Further in the present embodiment, an example has been explained, in which the magnetic resonance spectrum being measured according to the PRESS method sequence is used as the definitive spectrum or as the measured spectrum for creating each record 510 of the disease spectrum DB 500, but the sequence for acquiring the magnetic resonance spectrum is not limited to this example. By way of example, it may be a sequence of the 3D-CSI method that measures the magnetic resonance spectrum in units of multi-voxel, or a sequence of the EPSI method that is able to perform high-speed multi-voxel measurement.

In the present embodiment, the computer 109 of the MRI apparatus 100 is provided with the spectrum generator 210, the spectrum analyzer 220, the DB creator 230, and the candidate disease extractor 240, so as to create the magnetic resonance spectrum, analyze the magnetic resonance spectrum, create each record 510 to be registered in the disease spectrum DB 500, and extract to the candidate disease, but this is not the only example. In the case where the computer 109 of the MRI apparatus 100 is provided with an external device and a data transmit-receive function, the external device independent from the MRI apparatus 100 may be provided with at least one of the spectrum generator 210, the spectrum analyzer 220, the DB creator 230, and the candidate disease extractor 240, and perform the process according to this external device.

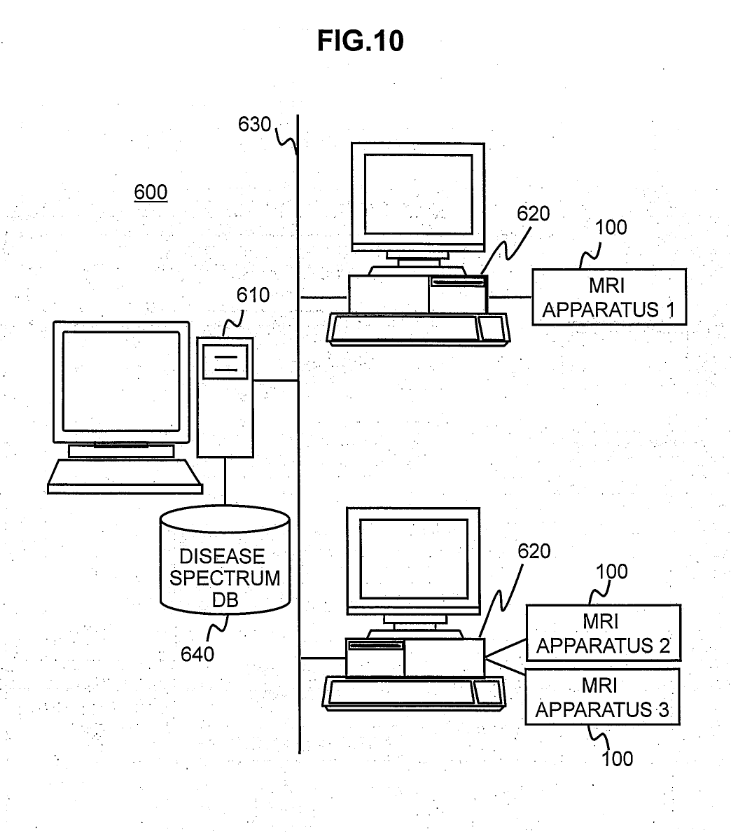

FIG. 10 illustrates an example of the system 600 in the case where the processes above are performed by a device other than the MRI apparatus 100. As illustrated, this system 600 is provided with a server 610 having the spectrum analyzer 220, the DB creator 230, and the candidate disease extractor 240, and plural clients 620 connected to the MRI apparatus 100. In this example here, it is assumed that each of the clients 620 is provided with the spectrum generator 210. The server 610 and each of the clients 620 are provided with communication interfaces, respectively, for performing data transmission and reception with an external device, and they are connected to each other via the communication line 630. The server 610 is provided with the storage device 640 for storing the disease spectrum DB being created, for instance.

It is to be noted that the storage device 640 for storing the disease spectrum DB may be provided with a communication interface, and connected to the communication line 630 in such a manner as independent of the server 610 and the clients 620. In this case, the server 610 and the clients 620 are allowed to access the storage device 640 via the communication line 630. In addition, the client 620 may be provided with at least one of the spectrum analyzer 220, the DB creator 230 and the candidate disease extractor 240. The server 610 may be provided with the spectrum generator 210.

It is to be noted that the configuration shown in the FIG. 10 above is just an example, and it is not limited to this configuration. By way of example, more than one server 610 may exist, and three or more clients 620 and four or more MRI apparatuses 100 may exist. In the example above, there is described the case where the communication line 630 is used to transmit the information, but transmission of the information may be performed through the use of a magnetic disk or an optical disk.

It is to be noted the MRI apparatus and system are assumed as having a function to create the disease spectrum DB by themselves. However, if the disease spectrum DB is standardized according to a consensus built by doctors, etc., in the specialized field, this may eliminate the need for the function to create the disease spectrum DB by the MRI apparatus and system by themselves. In other words, it is further possible to establish a system in which a standardized disease spectrum DB is placed on the cloud side in a computer utilization form on the basis of the network (the Internet, in particular) such as cloud computing, thereby downloading the disease spectrum DB in response to a request from the client side, and returning diagnostic support information (candidate disease list) in response to the measured spectrum data that is uploaded from the client side.

### Example

Hereinafter, an example of the present invention will be described.

Table 1 shows an example of the disease spectrum DB that was created from the definitive spectrum being acquired when a patient having a disease in the human head portion was assumed as the subject 101. The MRI apparatus being used was the MRI apparatus 100 (the static magnetic field strength was 1.5 tesla) as shown in FIG. 1A. The sequence being executed was the PRESS-method pulse sequence (TR/TE=2000 ms/136 ms) as shown in FIG. 3 above. The types of the disease targeted for creating the disease spectrum DB were four; abscess, glioblastoma, metastatic cancer, and meningioma.

It was further assumed that the definitive spectra were acquired with respect to each disease, the number of the definitive spectra being sufficient for creating the records in the disease spectrum DB. FIG. 6 as described above shows the procedure for the creation. Only the reliability index value (in this example here, the standard deviation rate % SD of each metabolite) was extracted and shown, as the registered value of each record in the disease spectrum DB.

### [Table 1]

TABLE 1

| | | | | | | | | |
|---|---|---|---|---|---|---|---|---|
| METABOLITE | NAA | Cr | Cho | Ins | Lac | Ala | Lip09 | Lip13 |

DISEASE ABSCESS

| | | | | | | | | |
|---|---|---|---|---|---|---|---|---|
| % SD | 19 | 19 | 15 | 50 | 856 | 134 | 15 | 12 |

DISEASE GLIOBLASTOMA

| | | | | | | | | |
|---|---|---|---|---|---|---|---|---|
| % SD | 13 | 12 | 7 | 30 | 724 | 76 | 18 | 10 |

DISEASE METASTATIC CANCER

| | | | | | | | | |
|---|---|---|---|---|---|---|---|---|
| % SD | 17 | 19 | 6 | 623 | 814 | 233 | 16 | 9 |

DISEASE MENINGIOMA

| | | | | | | | | |
|---|---|---|---|---|---|---|---|---|
| % SD | 17 | 19 | 6 | 623 | 814 | 233 | 16 | 9 |

Next, the PRESS-method pulse sequence (TR/TE=2000 ms/136 ms) as shown in FIG. 3 was executed in the same MRI apparatus 100 (the static magnetic field strength was 1.5 tesla), and the magnetic resonance spectrum obtained from the disease region (measured voxel size=8.0 cc=20 mm×20 mm×20 mm) in proximity to the temporal lobe of the human head portion was assumed as the measured spectrum targeted for the analysis. The aforementioned spectral analysis process was applied to this measured spectrum, and Table 2 shows the measured analysis data being obtained. In here, similar to the case above, only the standard deviation rate % SD of each metabolite is shown.

### [Table 2]

TABLE 2

| METABOLITE | NAA | Cr | Cho | Ins | Lac | Ala | Lip09 | Lip13 |
|---|---|---|---|---|---|---|---|---|
| % SD | 17 | 19 | 6 | 623 | 814 | 233 | 16 | 9 |

Then, through the use of the measured analysis data as shown in Table 2 and each record in the disease spectrum DB of the head portion as shown in Table 1 above, the processes from the step S1102 of the candidate disease extraction process according to the present embodiment were executed. In this case, the reference value B2 of the standard deviation rate % SD being used was 20.

As a result, the sum of difference DF of the standard deviation rate % SD for each disease was obtained as the following:

Difference accumulation (DF(abscess)) using the record in which the disease was abscess

DF(abscess)=√((19−11)²+(19−10)²+(15−5)²+(15−20)²+(12−8)²)=16.9

Difference accumulation (DF (glioblastoma)) using the record in which the disease was glioblastoma

DF(glioblastoma)=4√((13−11)²+(12−10)²+(7−5)²+(18−20)²+(10−8)²)=4.5

Difference accumulation (DF (metastatic cancer)) using the record in which the disease was metastatic cancer

DF(metastatic cancer)=√((17−11)²+(19−10)²+(6−5)²+(16−20)²+(9−8)²)=11.6

Difference accumulation (DF (meningioma)) using the record in which the disease was meningioma

DF(meningioma)=√((78−11)²+(92−10)²+(9−5)²+(356−20)²+(415−8)²)=538.3

The disease names are listed in the ascending order of the sum of difference (DF (disease)) values using the standard deviation rate % SD on a disease basis, as the following. Here, the value inside the parentheses represents the value of each sum of difference DF:

1) Glioblastoma (4.5)

2) Metastatic cancer (11.6)

3) Abscess (16.9)

4) Meningioma (538.3)

In this example here, it is found that the measured analysis data has the highest similarity to the record in which the disease is glioblastoma. Therefore, glioblastoma is considered as the candidate disease relating to this measured spectrum.

As explained so far, the MRI apparatus 100 of the present embodiment is provided with the computer 109 configured to apply an arithmetic processing to the nuclear magnetic resonance signal being acquired, and the monitor 110 configured to display a result of the arithmetic processing performed in the computer. The computer 109 is provided with the spectrum generator 210 configured to generate a magnetic resonance spectrum from the nuclear magnetic resonance signal, the spectrum analyzer 220 configured to analyze the magnetic resonance spectrum and calculate a reliability index being predetermined for each predetermined feature item as the analysis data, the candidate disease extractor 240 configured to use the disease spectrum DB 500 that registers the record 510 on a disease basis, the record 510 being created from definitive analysis data being the analysis data of at least one magnetic resonance spectrum that is diagnosed as a predetermined disease, so as to extract a candidate disease being estimated from the magnetic resonance spectrum as to which the disease is unknown, and displays a user, the candidate disease being estimated. The candidate disease extractor 240 uses the measured analysis data having a reliability degree that is indicated by the reliability index, equal to or higher than a predetermined value, among the measured analysis data being the analysis data of the magnetic resonance spectrum as to which the disease is unknown, so as to determine a similarity degree to the record 510 registered in the disease spectrum DB, and assumes the disease of the record 510 having a similarity degree equal to or higher than a predetermined value, as the candidate disease.

The computer 109 is further provided with the DB creator 230 configured to generate each record 510 of the disease spectrum DB 500, and the DB creator 230 may be configured such as creating the record 510 by using the definitive analysis data having the reliability degree that is indicated by the reliability index, equal to or higher than a predetermined value. At this time, the record 510 may be provided with a registered value for each feature item, and the registered value may be a statistical value of the reliability index of adopted data. It is further possible to determine such that the smaller is the sum of difference between the reliability index of the measured analysis data for each feature item and the registered value of the record 510, the higher is the similarity degree.

The present embodiment may be configured as the diagnostic support system 600 provided with the magnetic resonance imaging apparatus (MRI apparatus) 100 configured to generate a magnetic resonance spectrum from the nuclear magnetic resonance signal being acquired, and the server 610 configured to analyze the magnetic resonance spectrum obtained in the magnetic resonance imaging apparatus (MRI apparatus) 100. The server 610 is provided with the candidate disease extractor 230 configured to use the disease spectrum database 500 that registers the record on a disease basis, the record being generated by using definitive analysis data obtained from at least one of the magnetic resonance spectrum diagnosed definitely as a predetermined disease, so as to extract a candidate disease that is estimated from the magnetic resonance spectrum as to which the disease is unknown, and display to the user, the candidate disease being estimated. The definitive analysis data is provided with a predetermined reliability index for each predetermined feature item, obtained by analyzing at least one magnetic resonance spectrum being definitively diagnosed. The candidate disease extractor 230 uses measured analysis data having a reliability degree equal to or higher than a predetermined value, among the measured analysis data being the analysis data of the magnetic resonance spectrum as to which the disease is unknown, so as to determine a similarity degree to the record that is registered in the disease spectrum database, and assumes the disease of the record having the similarity degree equal to or higher than a predetermined value as the candidate disease.

The present embodiment may be implemented by a program that causes a computer to function as the candidate disease extracting unit (candidate disease extractor) 230 configured to use the disease spectrum database 500 in which the record is registered on a disease basis, the record being created by using definitive analysis data obtained from at least one magnetic resonance spectrum that is definitely diagnosed as relating to a predetermined disease, so as to extract a candidate disease being estimated from the magnetic resonance spectrum as to which the disease is unknown, display the candidate disease to the user. The definitive analysis data is provided with a predetermined reliability index for each feature item being predetermined, obtained by analyzing each of at least one magnetic resonance spectrum that is definitely diagnosed. The candidate disease extracting unit (candidate disease extractor) 230 uses the measured analysis data being a result of analyzing the magnetic resonance spectrum as to which the disease is unknown, and having a reliability degree that is indicated by the reliability index, equal to or higher than a predetermined value, so as to determine a similarity degree to the record that is registered in the disease spectrum database, and assumes the disease of the record having the similarity index equal to or higher than a predetermined value, as the candidate disease.

Therefore, according to the present embodiment, it is possible to automatically perform verification of the reliability degree of the magnetic resonance spectrum being acquired by the MRS imaging.

Upon creating the disease spectrum DB, it is automatically determined whether or not the analysis data obtained from the definitive spectrum with the definitive diagnosis is accepted, and whether or not the analysis data is adopted into the DB creation. Therefore, even when a doctor is not specialized in the field, he or she could construct easily the spectrum DB on a disease basis, from the definitive spectrum with a definitive diagnosis. Upon constructing the disease spectrum DB, there is no procedure to make a choice of data by a specialist, and therefore, it is possible not only to save time, but also to eliminate errors caused by differences in subjective views among the specialists, thereby allowing the precision of the constructed disease spectrum DB to be kept constant. In addition, the user is able to update the disease spectrum DB easily, and a high degree of flexibility is offered.

Similarity comparison with the data registered in the disease spectrum DB is performed using only the data that is determined as having a reliability degree equal to or higher than a predetermined value, among the analysis data of the measured spectrum. In addition, since the reliability index is used as a criterion for determining the similarity, this may enhance the probability to provide diagnostic support information with a higher degree of precision.

### EXPLANATION OF REFERENCES

100: MRI apparatus, 101: subject, 102: static magnetic field coil, 103: gradient coil, 104: shim coil, 105: transmitter coil, 106: receiver coil, 107: transmitter, 108: receiver, 109: computer, 110: monitor, 111: external storage device, 112: gradient magnetic field power supply, 113: shim power supply, 114: sequence controller, 115: input device, 120: MRI apparatus, 130: MRI apparatus, 210: spectrum generator, 220: spectrum analyzer, 230: DB creator, 240: candidate disease candidate disease extractor, 300: pulse sequence, 410: transverse image for positioning, 420: sagittal image for position reference, 430: coronal image for position reference, 441: the first slice, 442: the second slice, 443: the third slice, 450: volume of interest, 500: disease spectrum DB, 510: record, 520: disease name, 530: feature item, 540: registered value, 550: measured analysis data, 600: system, 610: server, 620: client, 630: communication line, 640: storage device, RF: radio frequency magnetic field, Gz: gradient magnetic field in the Z axis direction, Gy: gradient magnetic field in the Y axis direction, Gx: gradient magnetic field in the X axis direction, A/D: acquisition of signal, RF1: radio frequency magnetic field pulse, RF2: radio frequency magnetic field pulse, RF3: radio frequency magnetic field pulse, Gs11: slice selective gradient magnetic field pulse, Gr11: rephase (rephase) gradient magnetic field pulse, Gd21: dephase (dephasing) gradient magnetic field pulse, Gd21′: dephase (dephasing) gradient magnetic field pulse, Gd31: dephase (dephasing) gradient magnetic field pulse, Gd31′: dephase (dephasing) gradient magnetic field pulse, Gd22: dephase (dephasing) gradient magnetic field pulse, Gs22: slice selective gradient magnetic field pulse, Gd22′: dephase (dephasing) gradient magnetic field pulse, Gd32: dephase (dephasing) gradient magnetic field pulse, Gd32′: dephase (dephasing) gradient magnetic field pulse, Gd23: dephase (dephasing) gradient magnetic field pulse, Gd23′: dephase (dephasing) gradient magnetic field pulse, Gd33: dephase (dephasing) gradient magnetic field pulse, Gs33: slice selective gradient magnetic field pulse, Gd33′: Sig.1: nuclear magnetic resonance signal, TR: repetition time, TE: echo time, NAA: N-acetylaspartate acid signal, Cr: creatine signal, Cho: choline signal, Ins: inositol signal, Lac: lactate signal, Ala: alanine signal, Lip09: lipid signal having the peak at the frequency position of 0.9 ppm, Lip13: lipid signal having the peak at the frequency position of 1.3 ppm

What is claimed is:

1. A magnetic resonance imaging apparatus, comprising a computer configured to apply an arithmetic processing to a nuclear magnetic resonance signal being acquired, and a monitor configured to display a result of the arithmetic processing performed in the computer, wherein,
the computer comprises,
a spectrum generator configured to generate a magnetic resonance spectrum from the nuclear magnetic resonance signal,
a spectrum analyzer configured to analyze the magnetic resonance spectrum, and calculate a reliability index being predetermined for each predetermined feature item, as analysis data, and
a candidate disease extractor configured to use a disease spectrum database that registers a record on a disease basis, the record being created from definitive analysis data being the analysis data of at least one magnetic resonance spectrum that is diagnosed as a predetermined disease, so as to extract a candidate disease being estimated from the magnetic resonance spectrum as to which the disease is unknown, and display to a user the candidate disease being estimated,
the candidate disease extractor using measured analysis data having a reliability degree that is indicated by the reliability index being equal to or higher than a predetermined value, among the measured analysis data being the analysis data of the magnetic resonance spectrum as to which the disease is unknown, so as to determine a similarity degree to the record registered in the disease spectrum database, and assuming the disease of the record having the similarity degree equal to or higher than a predetermined value, as the candidate disease.

2. The magnetic resonance imaging apparatus according to claim 1, wherein,
the computer is further provided with a database creator configured to generate each record of the disease spectrum database, and
the database creator creates the record by using adopted data having the reliability degree that is indicated by the reliability index, equal to or higher than a predetermined value, among the definitive analysis data.

3. The magnetic resonance imaging apparatus according to claim 2, wherein,
the record comprises a registered value as to each of the feature item, and
the registered value is a statistical value of the reliability index of the adopted data.

4. The magnetic resonance imaging apparatus according to claim 3, wherein,
the smaller is a sum of squared difference between the reliability index of the measured analysis data for each feature item and the registered value of the record, the higher is the similarity degree.

5. The magnetic resonance imaging apparatus according to claim 1, wherein,
the spectrum analyzer further calculates as the analysis data, at least either of a concentration value and a signal intensity value, the feature item is at least either of a metabolite and a signal peak, and
the reliability index is a standard deviation rate of the concentration value, when the feature item is the metabolite, and the reliability index is a signal to noise ratio calculated from the signal intensity value, when the feature item is the signal peak.

6. A diagnostic support system comprising a magnetic resonance imaging apparatus configured to generate a magnetic resonance spectrum from a nuclear magnetic resonance signal being acquired, and a server configured to analyze the magnetic resonance spectrum obtained in the magnetic resonance imaging apparatus, wherein,
the server comprises a candidate disease extractor configured to use a disease spectrum database that registers a record on a disease basis, the record being generated by using definitive analysis data obtained from at least one of the magnetic resonance spectrum diagnosed definitely as a predetermined disease, so as to extract a candidate disease that is estimated from the magnetic resonance spectrum as to which the disease is unknown, and display to a user, the candidate disease being extracted, and
the definitive analysis data comprises a reliability index being predetermined for each predetermined feature item, being obtained by analyzing at least one magnetic resonance spectrum being definitively diagnosed, and
the candidate disease extractor uses measured analysis data having a reliability degree that is indicated by the reliability index, being equal to or higher than a predetermined value, among the measured analysis data being the analysis data of the magnetic resonance spectrum as to which the disease is unknown, so as to determine a similarity degree to the record registered in the disease spectrum database, and assumes the disease of the record having the similarity degree equal to or higher than a predetermined value, as the candidate disease.

7. The diagnostic support system according to claim 6, wherein,
the server further comprises a database creator configured to generate each record of the disease spectrum database, and
the database creator creates the record by using the definitive analysis data having the reliability degree that is indicated by the reliability index, equal to or higher than a predetermined value.

8. A program causing a computer to function as a candidate disease extracting unit configured to use a disease spectrum database in which a record is registered on a disease basis, the record being created by using definitive analysis data obtained from at least one magnetic resonance spectrum that is definitely diagnosed as relating to a predetermined disease, so as to extract a candidate disease being estimated from the magnetic resonance spectrum as to which the disease is unknown, and display the candidate disease to a user,
the definitive analysis data comprising a predetermined reliability index for each feature item being predetermined, obtained by analyzing each of at least one magnetic resonance spectrum that is definitely diagnosed,
the candidate disease extracting unit using measured analysis data having a reliability degree that is indicated by the reliability index, equal to or higher than a predetermined value, determining a similarity degree to the record that is registered in the disease spectrum database, and assuming the disease of the record having the similarity degree equal to or higher than a predetermined value, as the candidate disease.

9. The program according to claim 8, further causing the computer to function as a data base creating unit configured to generate each record of the disease spectrum database, by using the definitive analysis data having the reliability degree that is indicated by the reliability index, being equal to or higher than the predetermined value.
